(19) United States
(12) Patent Application Publication
(10) Pub. No.: US 2026/0297641 A1
(43) Pub. Date: Oct. 1, 2026

(54) METHOD FOR PRODUCING SOLUTION AND SOLUTION

(71) Applicant: HONDA MOTOR CO., LTD., Tokyo (JP)

(72) Inventors: Nozomi SHIOBARA, Tokyo (JP); Shigeru NAMIKI, Wako-shi (JP); Kenji MACHIDA, Wako-shi (JP); Tatsuhiko TANIYAMA, Tokyo (JP)

(21) Appl. No.: 19/633,045

(22) Filed: Mar. 30, 2026

(30) Foreign Application Priority Data
Mar. 31, 2025 (JP) 2025-059184

(51) Int. Cl. C12P 21/00; A01N 43/16; A01N 43/36; A01N 65/03; A01P 21/00; C12N 9/88; C12P 13/24; C12P 19/04

(52) U.S. Cl. CPC C12P 21/00; A01N 43/16; A01N 43/36; A01N 65/03; A01P 21/00; C12N 9/88; C12P 13/24; C12P 19/04; C12P 21/005; C12Y 402/01001

(57) ABSTRACT

A method for producing a solution obtained from a culture of an alga includes changing a ratio between a protein produced by the alga and a carbohydrate produced by the alga by adjusting a component of a culture solution in which to culture the alga and changing intracellular components of the alga. A solution obtained from a culture of an alga, the solution includes a protein produced by the alga and a carbohydrate produced by the alga.

### CROSS REFERENCE TO RELATED APPLICATIONS

This application claims the foreign priority benefit under 35 U.S.C. § 119 of Japanese patent application No. 2025-059184, filed on Mar. 31, 2025, the disclosure of which is incorporated herein by reference.

### BACKGROUND OF THE INVENTION

### 1. Field of the Invention

The present invention relates to a method for producing a solution and a solution.

### 2. Description of the Related Art

In agriculture, there are various issues such as increase in food demand due to the world population growth and the like and influence of the climate change on the productivity of crops. In view of this, biostimulants have been attracting attention. The biostimulants are a technology which alleviates damages of plants attributable to conditions of climates and soils by controlling abiotic stresses to plants and thus provides healthy plants. The includes, abiotic stress for example, drought damage, high-temperature injury, salt damage, cold-weather damage, frost damage, oxidative stress (damages due to active oxygen), physical injuries (damages from hail or wind), agent-induced damages due to pesticides, and the like.

For example, Patent Literature 1 discloses a heat tolerance or drying tolerance improving agent, a salt tolerance improving agent, or an activity improving agent for plants, including acetic acid or a salt thereof, or a solvate thereof and malic acid or a salt thereof, or a solvate thereof.

### PATENT LITERATURE

Patent Literature 1: JP2024-178407A

### SUMMARY OF THE INVENTION

Biostimulants having performances suitable for objects in accordance with the types of plants, environments, and the like have been demanded. However, it has conventionally been necessary to develop a product individually for an object in order to obtain a biostimulant having performance suitable for the object, which takes time and effort.

An object of the present application is to provide a method for producing a solution capable of easily obtaining a solution which can be used as a biostimulant having performance suitable for an object, and a solution which can be used as a biostimulant having performance suitable for an object.

To achieve the above-described object, a method for producing a solution obtained from a culture of an alga includes changing a ratio between a protein produced by the alga and a carbohydrate produced by the alga by adjusting a component of a culture solution in which to culture the alga and changing intracellular components of the alga.

In addition, a solution according to the present invention is a solution obtained from a culture of an alga, including a protein produced by the alga and a carbohydrate produced by the alga.

According to the method for producing a solution of the present invention, a solution which can be used as a biostimulant having performance suitable for an object can be easily obtained.

In addition, the solution of the present invention can be used as a biostimulant having performance suitable for an object.

### BRIEF DESCRIPTION OF THE DRAWINGS

FIG. 1 is a flowchart showing a method for producing a solution in an embodiment.

FIG. 2 is a graph relating to a total weight of thinned buds of each group in Examples.

FIG. 3 is a graph relating to a vertical width of the largest leaf in plants in Examples.

FIG. 4 is a graph relating to a lateral width of a leaf in Examples.

FIG. 5 is a graph relating to the numbers of leaves in Examples.

FIG. 6 is a graph relating to weights of edible parts in Examples.

### DETAILED DESCRIPTION OF THE EMBODIMENTS

Hereinafter, an embodiment of the present invention will be described in detail; however, the embodiment of the present invention is not limited to the embodiment described below.

First, a solution will be described, and then, a method for producing this solution will be described.

### Solution

A solution is obtained from a culture of an alga, and contains a protein produced by the alga and a carbohydrate produced by the alga. In addition, the solution preferably contains an amino acid produced by the alga.

The alga to be cultured includes, for example, algae of genus Chlamydomonas, and includes, for example, Chlamydomonas reinhardtii among genus Chlamydomonas. Chlamydomonas reinhardtii is a unicellular green alga. Chlamydomonas reinhardtii is living in fresh water of ponds, marshes, soils, and the like. For this reason, Chlamydomonas reinhardtii can be cultured in fresh water, and pH of the culture solution can be set to neutral. For this reason, a solution in which Chlamydomonas reinhardtii is cultured is suitable as a biostimulant to be fed to plants and soils.

A protein produced by the alga includes, for example, an iron assimilation protein, carbonic anhydrase, and glycoprotein, and one or more of these are preferably contained.

An iron assimilation protein has an effect of imparting tolerance to drought, high-temperature, and salt damages. A carbonic anhydrase has an effect of improving efficiency of photosynthesis by assisting fixation of carbon dioxide. This promotes the growth of a plant. In addition, since the carbonic anhydrase adjusts the intracellular pH balance and assists plants to normally function even under stressed conditions, the carbonic anhydrase has an effect of enhancing tolerance to environmental stresses such as dryness and high temperatures. A glycoprotein has an effect of enhancing tolerance to environmental stresses such as dryness and salt damage. In addition, a glycoprotein has an effect of reinforcing the immune system of plants and enhancing resistance to diseases and pests.

The carbohydrate produced by the alga includes, for example, monosaccharides such as galactose, and polysaccharides such as starch and pectin. These monosaccharides and polysaccharides have an effect of protecting plants from oxidation.

The amino acid produced by the alga includes, for example, hydroxyproline. The hydroxyproline has an effect of assisting plants to normally function even under stressed conditions by adjusting the intracellular osmotic pressure and keeping the stability of the cell membranes. In addition, the hydroxyproline has an effect of promoting the health and growth of plants as a whole by assisting the growth of roots. Moreover, the hydroxyproline also has an antioxidative action.

The solution is used as a biostimulant by being fed to at least one of a plant or a soil, for example. In the case of feeding the solution to a plant, the solution may be sprayed over the leaves and stems, for example. The solution may be diluted with water to adjust the concentrations of the components as appropriate. In addition, the amount of the solution to be fed to a plant or a soil may be adjusted in accordance with the type of the plant or a desired object as appropriate.

Next, a method for producing a solution will be described. The method for producing a solution is a method for producing a solution obtained from a culture of an alga, including changing a ratio between a protein produced by the alga and a carbohydrate produced by the alga by adjusting a component of a culture solution in which to culture the alga and changing intracellular components of the alga. In this way, the components of the solution to be obtained are adjusted.

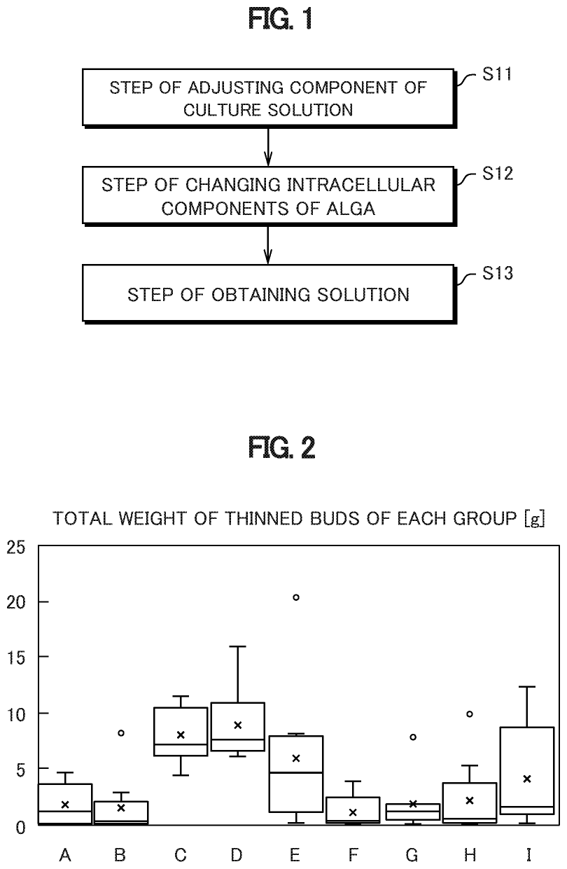

As shown in FIG. 1, in the present embodiment, the method for producing a solution will be described as including step S11 of adjusting a component of a culture solution, step S12 of changing intracellular components of an alga, and step S13 of obtaining a solution.

### (Step of Adjusting Component of Culture Solution)

The step S11 of adjusting a component of a culture solution is a step of adjusting a substance and content of a culture solution in which to culture an alga, to prepare a culture solution in which the component is adjusted.

In the step S11 of adjusting a component of a culture solution, for example, a desired component is added to pure water. Representative substances and contents (Final culture concentrations) of a culture solution are, for example, as follows.
Urea: 10 ppm or more and 10,000 ppm or less
Magnesium sulfate heptahydrate: 1.0 ppm or more and 1,000 ppm or less
Calcium chloride dihydrate: 0.1 ppm or more and 100 ppm or less
Dipotassium phosphate: 10 ppm or more and 10,000 ppm or less
Potassium dihydrogen phosphate: 10 ppm or more and 10,000 ppm or less
Zinc sulfate heptahydrate: 1.0 ppm or more and 1,000 ppm or less
Boric acid: 1.0 ppm or more and 1,000 ppm or less
Manganese chloride tetrahydrate: 0.1 ppm or more and 100 ppm or less
Iron sulfate heptahydrate: 0.1 ppm or more and 100 ppm or less
Copper sulfate pentahydrate: 0.1 ppm or more and 100 ppm or less
Ammonium heptamolybdate tetrahydrate: 0.1 ppm or more and 100 ppm or less

Besides, other general components used in culture of an alga can be added as appropriate.

The component of the culture solution to be adjusted includes at least one of nitrogen, phosphoric acid, or potassium. For example, the content of nitrogen can be adjusted by adjusting the content of urea. For example, by increasing the content of urea to increase the content of nitrogen, the protein is increased and the starch is reduced in the cells of the alga. On the other hand, by reducing the content of urea to reduce the content of nitrogen, the protein is reduced and the starch is increased in the cells of the alga. In this way, the ratio between the protein and the carbohydrate in the solution can be adjusted.

In addition, for example, by adjusting at least one of dipotassium phosphate or potassium dihydrogen phosphate, at least one of phosphoric acid or potassium can be adjusted.

Besides, these contents may be adjusted by adding at least one of nitrogen, phosphoric acid, or potassium to the culture solution.

### (Step of Changing Intracellular Components of Alga)

The step S12 of changing intracellular components of the alga is a step of changing intracellular components of the alga by culturing the alga.

In the step S12 of changing intracellular components of the alga, the alga is added to the culture solution in which the component has been adjusted, and cultured, for example, under conditions of 24 hours or more and 240 hours or less at 5° C. or more and 40° C. or less to change the intracellular components of the alga. In this way, the ratio between the protein and the carbohydrate in the cells of the alga is changed. Since the substances secreted by the alga to the outside of the cells are changed in this way, the composition of the solution changes. As the alga, there is Chlamydomonas reinhardtii, for example, as described above. In this way, a culture solution containing the components produced by the alga is prepared.

### (Step of Obtaining Solution)

The step S13 of obtaining a solution is a step of obtaining a solution from the culture solution containing the components produced by the alga after the culturing.

In the step S13 of obtaining a solution, for example, the alga is precipitated by centrifuging the culture solution after the culturing to separate the culture solution into the alga and a supernatant solution. This supernatant solution is taken out as the solution of the present embodiment. However, the culture solution after culturing the alga may be used as the solution as it is in accordance with a desired object or the like.

### EXAMPLES

Hereinafter, the present invention will be described in detail by using Examples; however, the present invention is not limited to these.

In the test in the present Examples, in order to verify the values of biostimulants (hereinafter, referred to as BS as appropriate) derived from the alga, the BS was applied to komatsuna, which is an evaluation crop generally used in agricultural institutes, to check the characteristics of expression effects. In addition, in the test, tendencies due to differences between a normal environment and a dry stress environment as well as tendencies due to differences in sprayed part and spraying frequency were checked.

First, Chlamydomonas reinhardtii was cultured in culture solutions containing components shown in Tables 1 to 3. The culturing was conducted at 25° C. However, for groups B, D, and F shown in Table 5, urea (UREA) was set to ¼ times, that is, 62.50 ppm. These culture solutions were centrifuged to collect supernatant solutions to prepare BS solutions. In addition, for groups H and I shown in Table 5, a new solution, which was a culture solution in which Chlamydomonas reinhardtii was not cultured, was used. Note that in groups B, D, and F, since UREA was ¼ times, the amount of the protein produced by Chlamydomonas reinhardtii was small, and the amount of starch produced by Chlamydomonas reinhardtii was large as compared with groups in which UREA was 1 time.

TABLE 1
UREA salt

| | | Final culture | |
|---|---|---|---|
| Substance name | Chemical formula | concentration | |
| Urea | CH₄N₂O | 250.00 | ppm |
| Magnesium sulfate | MgSO₄•7H₂O | 10.00 | ppm |
| heptahydrate | | | |
| Calcium chloride dihydrate | CaCl₂•2H₂O | 5.00 | ppm |

TABLE 2
Phosphate solution

| | | Final culture |
|---|---|---|
| Substance name | Chemical formula | concentration |
| Dipotassium phosphate | K₂HPO₄ | 720.00 ppm |
| Potassium dihydrogen | KH₂PO₄ | 360.00 ppm |
| phosphate | | |

TABLE 3
Hutner's trace elements

| | | Final culture | |
|---|---|---|---|
| Substance name | Chemical formula | concentration | |
| Zinc sulfate heptahydrate | ZnSO₄•7H₂O | 22.00 | ppm |
| Boric acid | H₃BO₃ | 11.40 | ppm |
| Manganese chloride | MnCl₂•4H₂O | 5.06 | ppm |
| tetrahydrate | | | |
| Iron sulfate heptahydrate | FeSO₄•7H₂O | 4.99 | ppm |
| Copper sulfate pentahydrate | CuSO₄•5H₂O | 1.57 | ppm |
| Ammonium heptamolybdate | (NH₄)₆Mo₇O₂₄•4H₂O | 1.10 | ppm |
| tetrahydrate | | | |

### <Test Method>

Black soil and red soil which did not contain nutrients were mixed well and were put into each pot. A groove of about 1 cm was formed with a finger, and seeds were planted averagely in each pot, and covered with the soil. Then, 600 ml of the BS of each condition was sprayed, and cultivation was started on a rooftop which was exposed uniformly to the sunlight with no shadow. As daily management, water was added to each pot from Monday to Friday to such an extent that the soil was sufficiently wetted. However, for dryness test pots of groups G and H shown in Table 5, water was added about once two days. For pots to which BS was sprayed five times and test pots for foliar spray in accordance with conditions, the spraying was executed in accordance with schedules shown in Table 4. In the state where the buds appeared to be about 2 cm, undesirable buds were thinned such that 6 buds were left in each pot. Once the komatsuna had grown to a size comparable to that of sold ones, cutting was carried out. The schedules of the test are shown in Table 4.

The spray amount was set 600 ml for the initial spray. In addition, from a result of hearing investigation to agricultural examination stations, the number of n was increased to 9 by using the pots such that each group had 9 pots to alleviate an influence of seed differences and the like.

### <Premise of Conditions>

The amount of N necessary for the pot test of komatsuna is 0.4 g in total in an area of 1/5000 of 1 a (are). When BS was sprayed, urea was added to control the amount of N such that the sprayed amount of N in total became 0.4 g. Since in the culture solutions after culturing Chlamydomonas reinhardtii, N decreased for the amount absorbed by Chlamydomonas reinhardtii, the amount of N decreased from the production amount of Chlamydomonas reinhardtii was calculated, and the amount of decrease was added to BS. In the case of 5 sprays of a 10-fold dilution, the amount of N was adjusted such that the amount of N became 0.4 g in total by the five sprays of a 10-fold dilution in addition to the amount added to compensate for the above-described decrease. For the adjustment of the amount of N, a UREA stock solution was used. Although the UREA stock solution contained Mg and Ca besides N, the contents of these were very small as compared with N, and were determined to have no influence, and the UREA stock solution was used.

### <Measurement Items>

The total weight of thinned buds, the vertical width of the largest leaf in the plant, the lateral width of the leaf, the number of leaves, and the weight of an edible part of each group were measured.

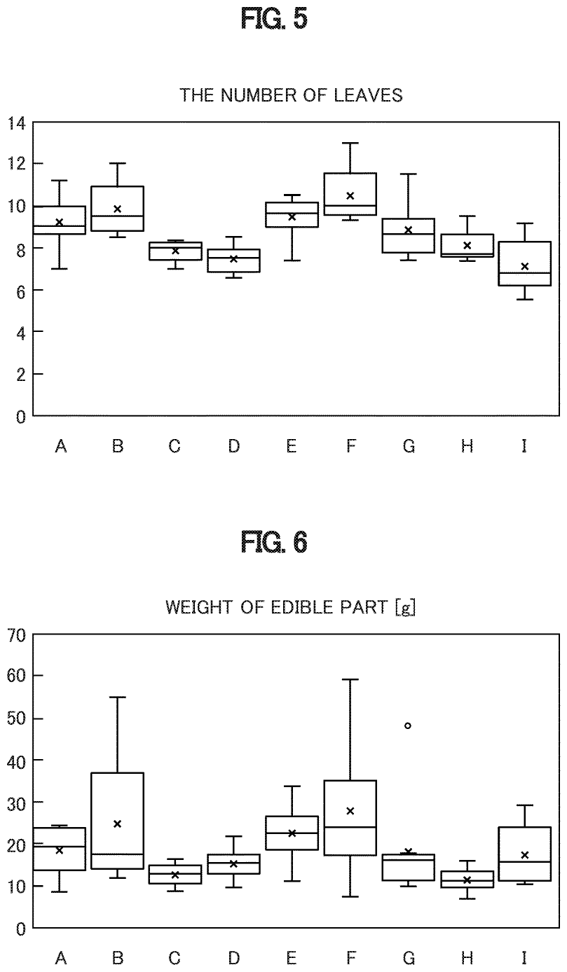

Test conditions for each sample are shown in Table 5. In addition, test results for the measurement items are shown in FIGS. 2 to 6. Note that in Table 5, groups A to G are Examples which satisfy the ranges of the present invention, and group H and I are Comparative Examples which did not satisfy the ranges of the present invention.

TABLE 5

| | | Water | | BS | Sprayed | Spraying |
|---|---|---|---|---|---|---|
| Group | Environment | content | BS culture | dilution | part | frequency |
| A | Rooftop | 200 ml | UREA × 1 | 1-fold | Soil | Only initial |
| | | daily | | | | soil spray |
| B | Rooftop | 200 ml | UREA × ¼ | 1-fold | Soil | Only initial |
| | | daily | | | | soil spray |
| C | Rooftop | 200 ml | UREA × 1 | 10-fold | Soil | Initial soil |
| | | daily | | | | spray + |
| | | | | | | additional 4 |
| | | | | | | sprays |
| D | Rooftop | 200 ml | UREA × ¼ | 10-fold | Soil | Initial soil |
| | | daily | | | | spray + |
| | | | | | | additional 4 |
| | | | | | | sprays |
| E | Rooftop | 200 ml | UREA × 1 | 1-fold | Soil + | Initial soil |
| | | daily | | | leaf | spray + 4 |
| | | | | | surface | foliar sprays |
| F | Rooftop | 200 ml | UREA × ¼ | 1-fold | Soil + | Initial soil |
| | | daily | | | leaf | spray + 4 |
| | | | | | surface | foliar sprays |
| G | Rooftop | About once | UREA × 1 | 1-fold | Soil | Only initial |
| | | two days | | | | soil spray |
| | | (dryness) | | | | |
| H | Rooftop | About once | New solution | 1-fold | Soil | Only initial |
| | | two days | | | | soil spray |
| | | (dryness) | | | | |
| I | Rooftop | 200 ml | New solution | 1-fold | Soil | Only initial |
| | | daily | | | | soil spray |

### <Consideration of Test Results>

From results of FIG. 2, groups C and D in which the dilution rate of BS was 10-fold were relatively favorable in terms of germination. From this, it was confirmed that in the case where s high emergence rate was required, it was favorable to set the dilution rate of BS to 10-fold.

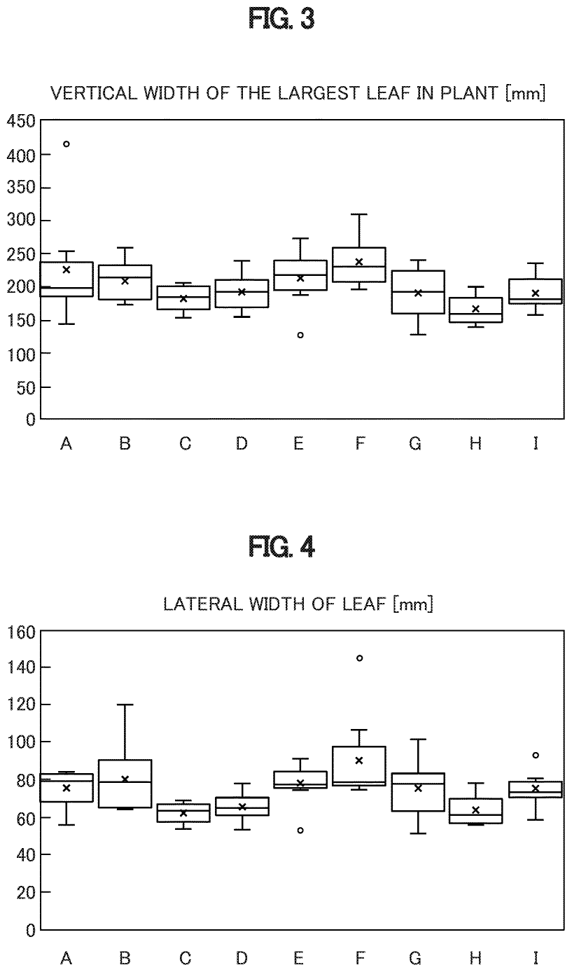

From results of FIGS. 3 to 6, it was confirmed that there was a tendency that it was favorable to use BS which was rich in starch produced by Chlamydomonas reinhardtii, and to set the dilution rate of BS to 1-fold, like groups B and F. In addition, from the results of FIGS. 3 to 6, it was confirmed that there was a tendency that foliar spray was favorable, like groups E and F. From the results of groups G and H of FIGS. 3 to 6, it was confirmed that there was a tendency that in a dry environment, it was favorable to use BS in which Chlamydomonas reinhardtii was cultured. Note that for groups C and D in which the dilution rate of BS was 10-fold, from the fact that since the germination of the thinned buds was relatively favorable, the left buds got little sunlight, and the left buds were small, it is considered that the sizes of the leaves, the number of leaves, and the weights of edible parts tended to be relatively small.

As described above, according to the present embodiment, a solution useful as a biostimulant can be obtained. Then, by adjusting the components of a culture solution of an alga, a solution having desired effects can be obtained. In addition, this solution enables a plant to express desired effects.

Although the modes for carrying out the invention have been more specifically described, the gist of the present invention is not limited by these descriptions, and the present invention shall be construed broadly based on the description of claims. In addition, those obtained by conducting various changes, modifications, and the like based on these descriptions are also encompassed in the gist of the present invention.

For example, in the embodiment, the ratio between the protein and the carbohydrate in the cells of the alga was changed by adjusting the content of urea of the culture solution. However, for example, the ratio between the protein and the carbohydrate in the cells of the alga may be changed by adjusting the content of another substance including nitrogen in the culture solution.

In addition, as another embodiment, the solution may contain a protein produced by an alga and an amino acid produced by the alga. If the solution contains a protein and an amino acid produced by an alga as major components, the solution can be used as a biostimulant for a desired object. In addition, an alga may be caused to produce at least one of a protein, an amino acid, a carbohydrate, or another substance by adjusting a component of a culture solution in which the alga is to be cultured. In addition, a ratio of these produced by an alga may be changed by adjusting a component of a culture solution in which the alga to be is cultured. The types of a protein, an amino acid, and a carbohydrate are as described in the embodiment.

It is understood that the foregoing description is that of the preferred embodiments of the invention and that various changes and modifications may be made thereto without departing from the spirit and scope of the invention as defined in the appended claims.

What is claimed is:

1. A method for producing a solution obtained from a culture of an alga, the method comprising:
changing a ratio between a protein produced by the alga and a carbohydrate produced by the alga by adjusting a component of a culture solution in which to culture the alga and changing intracellular components of the alga.

2. The method according to claim 1, wherein
the alga is Chlamydomonas reinhardtii.

3. The method according to claim 1, wherein
the component of the culture solution is urea.

4. The method according to claim 1, wherein
the protein includes at least one of an iron assimilation protein, a carbonic anhydrase, or a glycoprotein.

5. The method according to claim 1, wherein
the carbohydrate is a polysaccharide.

6. The method according to claim 1, wherein
a component produced by the alga includes an amino acid.

7. The method according to claim 6, wherein
the amino acid is hydroxyproline.

8. A solution obtained from a culture of an alga, the solution comprising:
a protein produced by the alga; and
a carbohydrate produced by the alga.

9. The solution according to claim 8, wherein
the alga is Chlamydomonas reinhardtii.

10. The solution according to claim 8, wherein
the protein includes at least one of an iron assimilation protein, a carbonic anhydrase, or a glycoprotein.

11. The solution according to claim 8, wherein
the carbohydrate is a polysaccharide.

12. The solution according to claim 8 further comprising an amino acid produced by the alga.

13. The solution according to claim 12, wherein
the amino acid is hydroxyproline.

14. The method according to claim 2, wherein
the component of the culture solution is urea.

15. The method according to claim 2, wherein
the protein includes at least one of an iron assimilation protein, a carbonic anhydrase, or a glycoprotein.

16. The method according to claim 2, wherein
the carbohydrate is a polysaccharide.

17. The method according to claim 2, wherein
a component produced by the alga includes an amino acid.

18. The solution according to claim 9, wherein
the protein includes at least one of an iron assimilation protein, a carbonic anhydrase, or a glycoprotein.

19. The solution according to claim 9, wherein
the carbohydrate is a polysaccharide.

20. The solution according to claim 9 further comprising an amino acid produced by the alga.
